(19) United States
(12) Patent Application Publication
(10) Pub. No.: US 2006/0178656 A1
(43) Pub. Date: Aug. 10, 2006

(54) Treatment tool for endoscope

(75) Inventors: Noriyuki Sugita, Saitama (JP); Satoshi Kidooka, Saitama (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/349,083

(22) Filed: Feb. 8, 2006

(30) Foreign Application Priority Data
Feb. 9, 2005 (JP) 2005-032392
Mar. 3, 2005 (JP) 2005-058475
Mar. 3, 2005 (JP) 2005-058476

(51) Int. Cl. A61B 17/00

(52) U.S. Cl. 606/1

(57) ABSTRACT

A treatment tool for an endoscope, having a flexible sheath being made of electrically insulating material to be inserted through an instrument channel of the endoscope, an operation wire, which is adapted to be advanced and retracted in an axial direction of the sheath inside the sheath, and a treatment instrument, which is arranged at a distal portion of the treatment tool and is adapted to be protruded and retracted from a distal end of the sheath, is provided. The operation wire is connected to the treatment instrument via a connecting member having a distal surface. The sheath includes a stopper surface at the distal end thereof. The stopper surface is inclined with respect to the distal surface of the connecting member when the connecting member is installed in the sheath. An orientation of the treatment instrument is changed according to the inclination of the stopper surface by having the distal surface of the connecting member pressed against the stopper surface when the operation wire is pressed from a proximal end of the sheath toward the distal end of the sheath.

### BACKGROUND OF THE INVENTION

The present invention relates to a treatment tool for an endoscope.

Conventionally, various types of treatment tools for endoscopes have been provided, including a tip portion as a treatment instrument that is adapted to be protruded and retracted from a tip end of a sheath being inserted through an instrument channel of the endoscope. The tip portion is protruded and retracted by advancing and retracting an operation wire arranged in the sheath in an axial direction thereof. An example of such a treatment tool is disclosed in Japanese Patent Provisional Publication No. 2002-153484.

The above referenced treatment tool is not provided with a bending mechanism to control an orientation of the tip portion in an arbitrary direction by a remote operation from a user, however, such a mechanism is desirable to have the tip portion lead in a targeted area for the treatment.

Japanese Patent Provisional Publications No. HEI9-262239 and No. HEI11-42232 disclose treatment tools with such bending mechanisms. However, tip portions of these treatment tools are not configured to be protruded and retracted from distal ends of sheaths. Further, such treatment tools are required to have additional operation wires to bend distal portions inside the sheaths, which make the structures the operations of the treatment tools complicated.

In this regard, a treatment tool having a less complicated mechanism to control the orientation of the tip portion may be provided. The operation wire and the tip portion of such an treatment tool may have a connecting portion, wherein the operation wire and tip portion are connected to each other. The connecting portion may have a front surface, which is faced to the distal end of the sheath, and a sheath may have a stopper member with a rear surface at the distal end thereof. The rear surface may be inclined with respect to the front surface of the connecting portion. With this configuration, the orientation of the tip portion may be changed correspondingly to the inclination of the rear surface of the stopper member by having the front surface of the connecting member pressed against the rear surface of the stopper member when the operation wire is pressed from a proximal end toward the distal end of the sheath.

It should be noted that the treatment tool with the above-described configuration may require the operation wire to be substantially resilient to press the connecting portion forwardly. However, when the operation wire is too resilient, it may be difficult to control the orientation of the tip portion as the orientation is determined by the deformation of the operation wire being pressed.

### SUMMARY OF THE INVENTION

The present invention is advantageous in that a treatment tool for an endoscope capable of being controlled to orient in an arbitrary direction securely in a less complicated operation is provided.

According to an aspect of the present invention, a treatment tool for an endoscope, having a flexible sheath being made of electrically insulating material to be inserted through an instrument channel of the endoscope, an operation wire, which is adapted to be advanced and retracted in an axial direction of the sheath inside the sheath, and a treatment instrument, which is arranged at a distal portion of the treatment tool and is adapted to be protruded and retracted from a distal end of the sheath, is provided. The operation wire is connected to the treatment instrument via a connecting member having a distal surface. The sheath includes a stopper surface at the distal end thereof. The stopper surface is inclined with respect to the distal surface of the connecting member when the connecting member is installed in the sheath. An orientation of the treatment instrument is changed according to the inclination of the stopper surface by having the distal surface of the connecting member pressed against the stopper surface when the operation wire is pressed from a proximal end of the sheath toward the distal end of the sheath.

Optionally, the treatment instrument may be formed to be elongated. The sheath may be provided with a stopper member, of which rear surface defines the stopper surface, at the distal end thereof. The stopper member may be provided with a groove, of which direction corresponds to the orientation of the treatment instrument being changed, so that the treatment instrument can be guided in the groove.

Optionally, a cutout may be formed on the distal end of the sheath to be integrated with the groove.

Optionally, a cross-sectional area of a channel in which the operation wire is arranged may be formed to have an elongated shape in parallel with a plane, in which the orientation of the treatment tool is changed, at least at a distal portion of the sheath so that the operation wire can be deformed therein.

According to an aspect of the present invention, a treatment tool for an endoscope, having a flexible sheath being made of electrically insulating material to be inserted through an instrument channel of the endoscope, an operation wire, which is adapted to be advanced and retracted in an axial direction of the sheath inside the sheath, and a treatment instrument, which is arranged at a distal portion of the treatment tool and is adapted to be protruded and retracted from a distal end of the sheath, is provided. The operation wire is connected to the treatment instrument via a connecting member having a distal surface. The sheath includes a stopper surface at a distal portion thereof. The stopper surface is inclined with respect to the distal surface of the connecting member when the connecting member is installed in the sheath. The distal portion of the sheath includes a groove, in which the treatment instrument is guided, at a position corresponding to a rear end of the stopper surface with respect to a diameter of the distal portion of the sheath. The rear end of the stopper surface is a closest point to the distal surface of the connecting member. A depth of the groove is formed to be smaller than a perpendicular length from the rear end of the stopper surface to an axis of the sheath. An orientation of the treatment instrument is changed according to the inclination of the stopper surface by having the distal surface of the connecting member pressed against the stopper surface when the operation wire is pressed from a proximal end of the sheath toward the distal end of the sheath.

Optionally, the groove may be formed to allow the treatment instrument to protrude outwardly from the distal end of the sheath in parallel with the axis of the sheath.

According to an aspect of the present invention, a treatment tool for an endoscope, having a flexible sheath being made of electrically insulating material to be inserted through an instrument channel of the endoscope, an operation wire, which is adapted to be advanced and retracted in an axial direction of the sheath inside the sheath, and a treatment instrument, which is arranged at a distal portion of the treatment tool and is adapted to be protruded and retracted from a distal end of the sheath, is provided. The operation wire is connected to the treatment instrument via a connecting member having a distal surface. The sheath includes a stopper surface at a distal portion thereof. The stopper surface is inclined with respect to the distal surface of the connecting member when the connecting member is installed in the sheath. An orientation of the treatment instrument is changed according to the inclination of the stopper surface by having the distal surface of the connecting member pressed against the stopper surface when the operation wire is pressed from a proximal end of the sheath toward the distal end of the sheath. The operation wire includes a proximal portion and a distal portion that is formed with more flexible wire than a wire forming the proximal portion.

According to an aspect of the present invention, a treatment tool for an endoscope, having a flexible sheath being made of electrically insulating material to be inserted through an instrument channel of the endoscope, an operation wire, which is formed with a core wire and a plurality of peripheral wires. The plurality of peripheral wires are twisted around the core wire. The operation wire is adapted to be advanced and retracted in an axial direction of the sheath inside the sheath. The treatment tool further includes a treatment instrument, which is arranged at a distal portion of the treatment tool and is adapted to be protruded and retracted from a distal end of the sheath. The operation wire includes a distal surface, from which the treatment instrument is extended. The sheath includes a stopper surface at a distal portion thereof. The stopper surface is inclined with respect to the distal surface of the operation wire when the operation wire is installed in the sheath. An orientation of the treatment instrument is changed according to the inclination of the stopper surface by having the distal surface of the operation wire pressed against of the stopper surface when the operation wire is pressed from a proximal end of the sheath toward the distal end of the sheath. The core wire is elongated at a distal portion thereof to form the treatment instrument.

### BRIEF DESCRIPTION OF THE DRAWINGS

FIG. 1 shows a plane view of an entire configuration of a treatment tool according to embodiments of the invention.

FIG. 2 shows a cross-sectional side view of the entire configuration of the treatment tool according to the embodiments of the invention.

FIG. 3 shows a cross-sectional side view of a distal portion of the treatment tool according to a first embodiment of the invention.

FIG. 4 shows a cross-sectional view of the distal portion of the treatment tool taken along the line IV-IV in FIG. 3 according to the first embodiment of the invention.

FIG. 5 shows an exploded view of a stopper member and a sheath of the treatment tool according to the first embodiment of the invention.

FIG. 6 shows a cross-sectional side view of the distal portion of the treatment tool with an electrode biased at an angle according to the first embodiment of the invention.

FIG. 7 shows a cross-sectional side view of a distal portion of the treatment tool according to a second embodiment of the invention.

FIG. 8 shows a cross-sectional view of the distal portion of the treatment tool taken along the line VIII-VIII in FIG. 7 according to the second embodiment of the invention.

FIG. 9 shows an exploded view of a stopper member and a sheath of the treatment tool according to the second embodiment of the invention.

FIG. 10 shows a cross-sectional side view of a distal portion of the treatment tool according to a third embodiment of the invention.

FIG. 11 shows a cross-sectional view of the distal portion of the treatment tool taken along the line XI-XI in FIG. 10 according to the third embodiment of the invention.

FIG. 12 shows an exploded view of a stopper member and a sheath of the treatment tool according to the third embodiment of the invention.

FIG. 13 shows a cross-sectional side view of the distal portion of the treatment tool with an electrode biased at an angle according to the third embodiment of the invention.

FIG. 14 shows a cross-sectional side view of a distal portion of the treatment tool according to a fourth embodiment of the invention.

FIG. 15 shows a cross-sectional side view of a distal portion of the treatment tool according to a fifth embodiment of the invention.

FIG. 16 shows a cross-sectional side view of the distal portion of the treatment tool with an electrode biased at an angle according to the fifth embodiment of the invention.

FIG. 17 shows a perspective view of the electrode and an operation wire of the treatment tool according to the fifth embodiment of the invention.

### DETAILED DESCRIPTION OF THE EMBODIMENTS

Hereinafter, referring to the accompanying drawings, a treatment tool according to illustrative embodiments of the invention will be described.

### First Embodiment

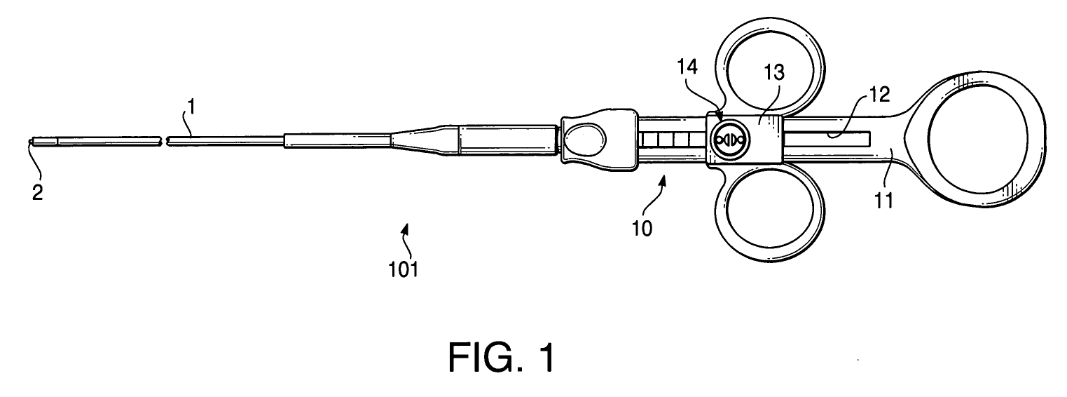

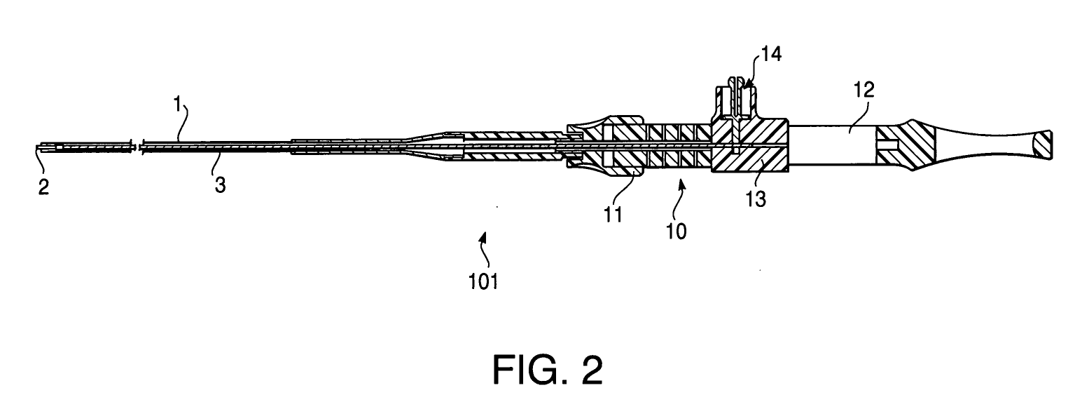

FIG. 1 shows a plane view of an entire configuration of a treatment tool 101 for an endoscope according to embodiments of the invention. FIG. 2 shows a cross-sectional side view of the entire configuration of the treatment tool 101 according to the embodiments of the invention.

The treatment tool 101 includes a flexible tubular sheath 1, which is made of an electrically insulated material, for example polytetrafluoroethylene. The sheath 1 is adapted to be inserted through an instrument channel (not shown) of the endoscope.

The treatment tool 101 further includes a rod-like high-frequency electrode 2 as a treatment instrument, which is adapted to be protruded outwardly and retracted inwardly via a resilient operation wire 3 by an operation from a user. The operation wire 3 consists of a plurality of thin wires that are twisted to form a single line wire, and is arranged in a lumen of the treatment tool 101 in parallel with an axis of the sheath 1.

The treatment tool 101 is provided with an operation unit 10 at a proximal end thereof. The operation unit 10 includes an operation shaft 11, which is connected to the proximal end of the sheath 1, a slit 12, which is formed in an axial direction of the operation shaft 11, and a slidable portion 13, which is adapted to slide along the slit 12. The slidable portion 13 is connected to a proximal end of the operation wire 3, which is advanced and retracted in the axial direction of the operation shaft 11 inside the sheath 11 with the slidable portion 13 being slid in the axial direction by the user.

The slidable portion 13 is further provided with a terminal 15, to which a power supplying cable (not shown) is connected, so that electrical current with high-frequency can be supplied to the operation wire 3.

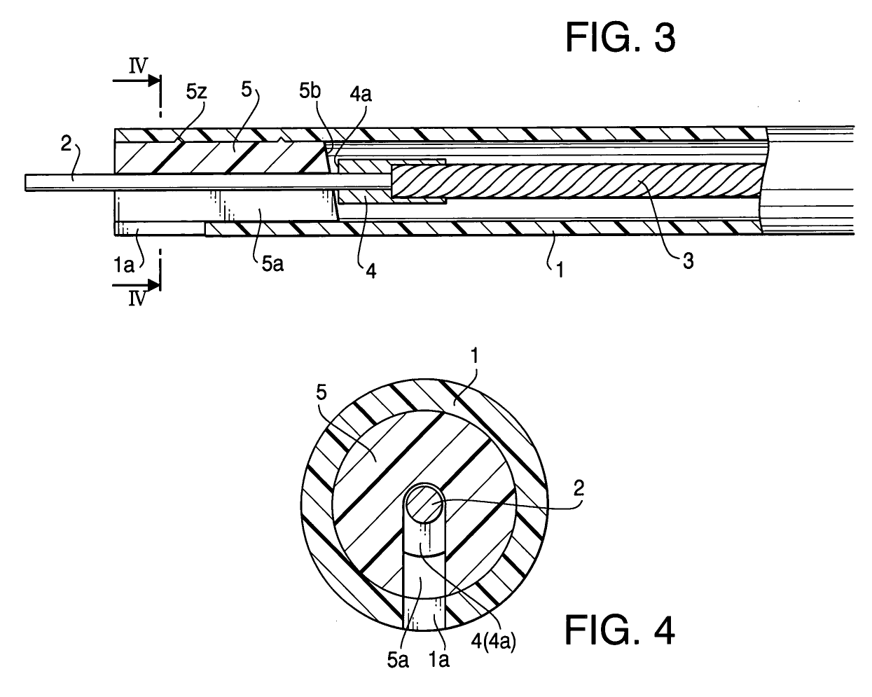

FIG. 3 shows a cross-sectional side view of a distal portion of the treatment tool 101 according to the first embodiment of the invention. A connecting member 4, made of electrically conductive metal, connects a distal end of the operation wire 3 with a proximal end of the electrode 2. The connecting member 4 is provided with two holes, which are in communication with each other at an axially substantially intermediate point of the connecting member 4. A diameter of one of the holes corresponds to an diameter of the electrode 2, so that the proximal end of the electrode 2 is to be inserted therein. A diameter of the other of the holes corresponds to an diameter of the operation wire 3, so that the distal end of the operation wire 3 is to be inserted therein. The electrode 2 and the operation wire 3 are then securely fixed to the connecting member 4 with silver brazing and the like, thus the operation wire 3 and the electrode 2 are connected.

The sheath 1 is further provided with a stopper member 5, which is made of an electrically insulated material, at the distal end of thereof. The stopper member 5 is formed to fit tightly to an inner diameter of the sheath 1, and is provided with a projection 5z on an outer periphery so that the projection 5z is wedged to an inner periphery of the sheath 1 and the stopper member 5 does not drop off. It should be noted that stopper member 5 may be screwed to the sheath 1, or may be fixed to the sheath 1 in other known method.

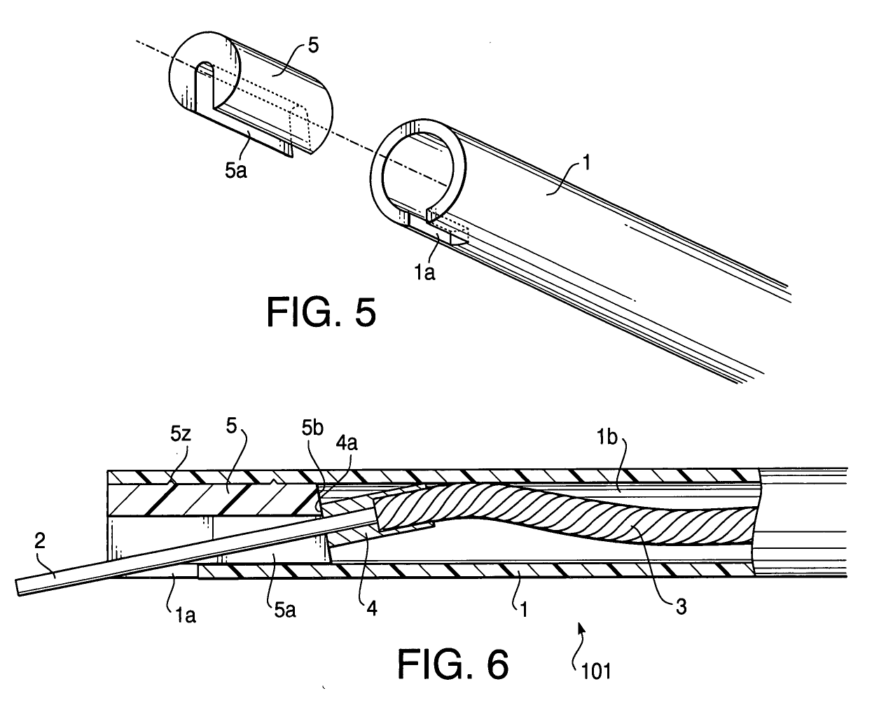

FIG. 4 shows a cross-sectional view of the distal portion of the treatment tool 101 taken along the line IV-IV in FIG. 3 according to the first embodiment of the invention. FIG. 5 shows an exploded view of the stopper member 5 and the sheath 1 of the treatment tool 101 according to the first embodiment of the invention.

The stopper member 5 is provided with a groove 5a in parallel with an axial direction of the stopping member 5. The groove 5a is formed to have a width that is substantially greater than the diameter of the electrode 2 and is substantially smaller than a diameter of the connecting member 4. Meanwhile, the sheath 1 is provided with a cutout 1a at the distal end thereof, which is in parallel with the axis of the sheath 1 and is formed to be integrated with the groove 5a when the stopper member 5 is installed to the sheath 1.

When the operation wire 3 is advanced via the operation unit 10 toward the distal end of the sheath 1, a front surface 4a of the connecting member 4 is in contact with a rear surface 5b of the stopper member 5. The rear surface 5b of the stopper member 5 is formed to be inclined with respect to the axis of the sheath 1, while the front surface 4a of the connecting member 4 is formed to be perpendicular to the axis of the sheath 1, so that an area on the rear surface 5b corresponding to the groove 5a is most projected toward the proximal end of the sheath 1. Therefore, the rear surface 5b is formed to be inclined with the axis of the sheath 1. It should be noted the front surface 4a of the connecting member 4 may be formed to be inclined when the rear surface 5b is formed to be perpendicular to the axis of the sheath 1.

FIG. 6 shows a cross-sectional side view of the distal portion of the treatment tool 101 with the electrode 2 biased at an angle according to the first embodiment of the invention. With the above-described configuration, when the operation wire 3 is pressed toward the distal end of the sheath 1 from the position indicated in FIG. 3 and the front surface 4a of the connecting member 4 is pressed against the stopper member 5, the connecting member 6 is turned at a small angle so that an entire area of the front surface 4a is in contact with the rear surface 5b, as shown in FIG. 6. Accordingly, the distal portion of the operation wire 3 that is in connection with the connecting member 4 is curved, and the electrode 2 that is also in connection with the connecting member 4 is biased in the groove 5a of the stopper member 5 and the cutout 1a of the sheath 1.

It should be noted that the inclination of the rear surface 5b and the groove 5a are formed to correspond each other, so that the orientation of the electrode 2 when the front surface 4a is pressed against the rear surface 5b and the orientation the groove 5a that guides the electrode 4a are coincided with each other, and the orientation of the electrode 2 can be changed smoothly along the groove 5a.

The orientation of the electrode 4 returns to the initial orientation, which is in parallel with the axial direction of the sheath 1, when the connecting member 6 is released from the pressing force. Thus, the orientation of the electrode 4 can be arbitrarily changed with the operation to the operation wire 5 that can be also used to for protruding and retracting the electrode 4.

### Second Embodiment

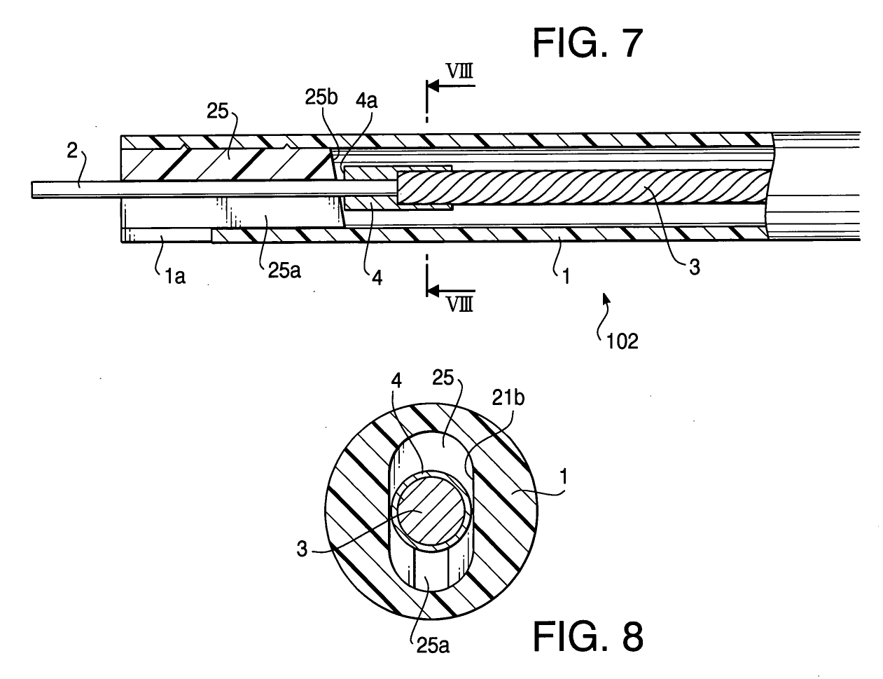

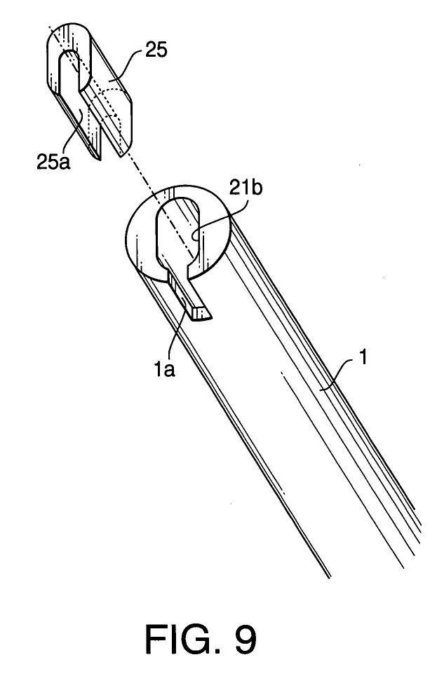

FIG. 7 shows a cross-sectional side view of a distal portion of the treatment tool 102 according to a second embodiment of the invention. FIG. 8 shows a cross-sectional view of the distal portion of the treatment tool 102 taken along the line VIII-VIII in FIG. 7 according to the second embodiment of the invention. FIG. 9 shows an exploded view of a stopper member 25 and a sheath 1 of the treatment tool 102 according to the second embodiment of the invention. In this and the following embodiments, configurations corresponding to the configuration of the first embodiment is referred to by the identical reference numerals, and description of those is omitted.

An operation wire 3 is arranged in a lumen 21b, and the lumen 21b is formed to have a cross-sectional area of an elongated shape, for example an oval, in parallel with a plane, in which an orientation of an electrode 4 is changed. Accordingly, the stopper member 25 is formed to have a cross-sectional area of an oval (see FIG. 9).

With the above-described configuration, when a connecting member 4 is pressed against the stopper member 25 and the operation wire 3 is deformed at a distal portion thereof, the orientation of the electrode 2 is changed, however, the orientation is restricted in the lumen 21b, and the electrode 2 is more steadily moved to change the orientation thereof. It should be noted that the lumen 21b may not have the cross-sectional area of an elongated shape for an entire length of the sheath 1, but may have the cross-sectional area of the elongated shape at least at the distal portion of the sheath 1.

It should be noted that a front surface 4a of the connecting member 4 may be formed to be inclined and a rear surface 25b of the stopper member 25 may be formed to be perpendicular to the axis of the sheath 1 for carrying out the invention.

It should be further noted that the electrode 2 may not necessarily be configured to be protruded toward the distal end of the treatment tools described above, but may be configured to be protruded in other direction. It should be also noted that the invention may be applied to a treatment tool that is not supplied with high-frequency electrical current.

### Third Embodiment

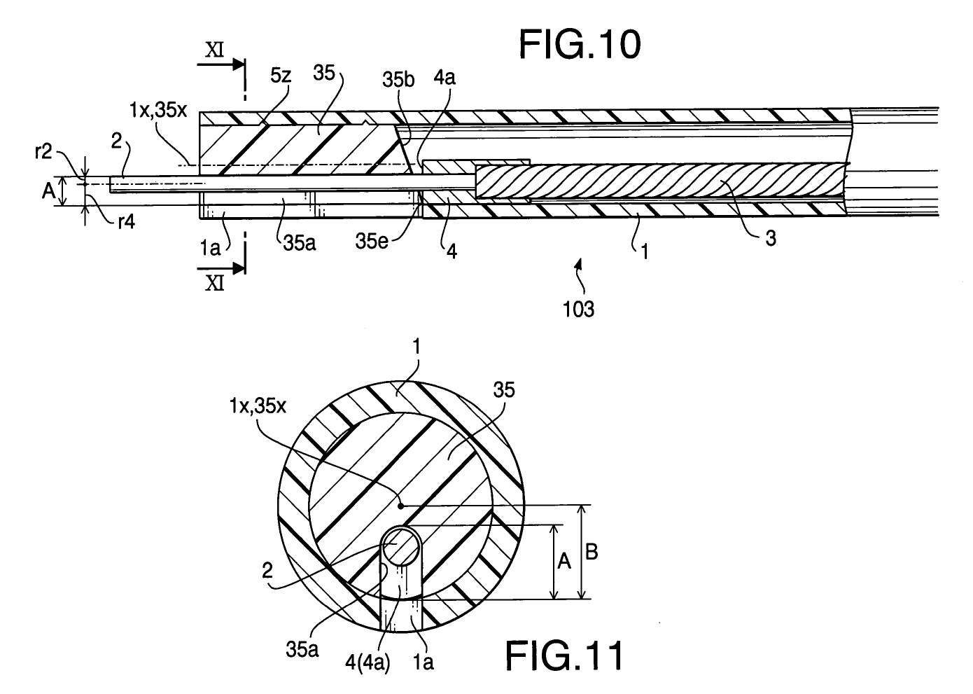

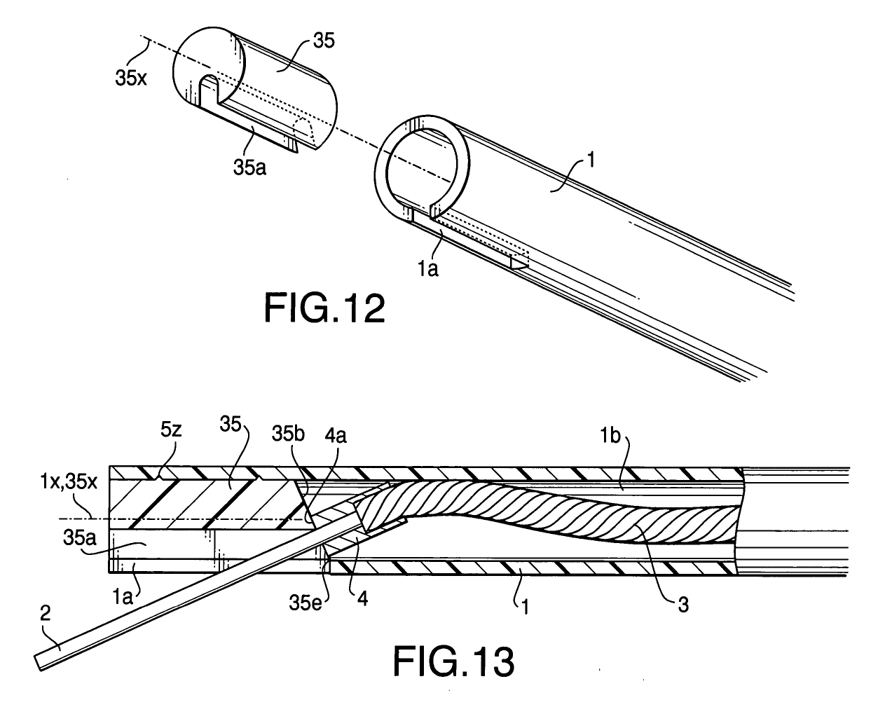

FIG. 10 shows a cross-sectional side view of a distal portion of the treatment tool 103 according to a third embodiment of the invention. FIG. 11 shows a cross-sectional view of the distal portion of the treatment tool 103 taken along the line XI-XI in FIG. 10 according to the third embodiment of the invention. FIG. 12 shows an exploded view of a stopper member 35 and a sheath 1 of the treatment tool 103 according to the third embodiment of the invention. FIG. 13 shows a cross-sectional side view of the distal portion of the treatment tool 103 with an electrode biased at an angle according to the third embodiment of the invention. It should be noted that an axis 1x of the sheath 1 and an axis 35x of the stopper member 35 coincides with each other when the stopper member 35 is installed to the sheath 1.

The stopper member 35 is provided with a groove 35a, which is formed to have a depth A, at a position corresponding to a rear end 35e of the stopper member 35 with respect to a diameter of the stopper member 35. The rear end 35e is a point closest to a front surface 4a of a connecting member 4 when the connecting member 4 is installed in the sheath 1. The depth A of the groove 35a is formed to be smaller than a perpendicular length B from the rear end 35e to the axis 35x (i.e., the axis 1x) in FIG. 11 (i.e., a radius of the stopper member 35).

Further, the depth A is formed to be greater than or equal to a sum of a radius r4 of the connecting member 4 and a radius 2r of the electrode 2 (i.e., A≧r4+r2). Therefore, when the electrode 2 is inserted through the groove 35a, the electrode 2 maintains parallel to the sheath 1 when an operation wire 3 is advanced forwardly but the connecting member 4 is not pressed against the stopper member 35 forcibly enough to deform the operation wire 3. When the connecting member 4 is pressed against the stopper member 35 forcibly, however, the operation wire 3 is deformed and an orientation of the electrode 2 is changed.

With the above-described configuration, the front surface 4a of the electrode 4 is pressed to a rear surface 35b of the stopper member 5 at a lower-side position in FIG. 13 that is closer to the rear end 35e (see also FIG. 6). As shown in FIG. 13, therefore the operation wire 3 can be deformed in relatively small space in the distal portion of the lumen 1b, and the lumen 1b can be effectively used to change the orientation of the electrode 2 at a greater angle with respect to the axis of the electrode 2. When the depth A of the groove 35a is formed to be substantially equivalent to the sum of the radius r4 of the connecting member 4 and the radius 2r of the electrode 2 (i.e., A=r4+r2), the electrode 2 can be oriented at a maximum angle with respect to the axis 1x of the sheath 1 allowed in the distal portion of the lumen 1b.

### Fourth Embodiment

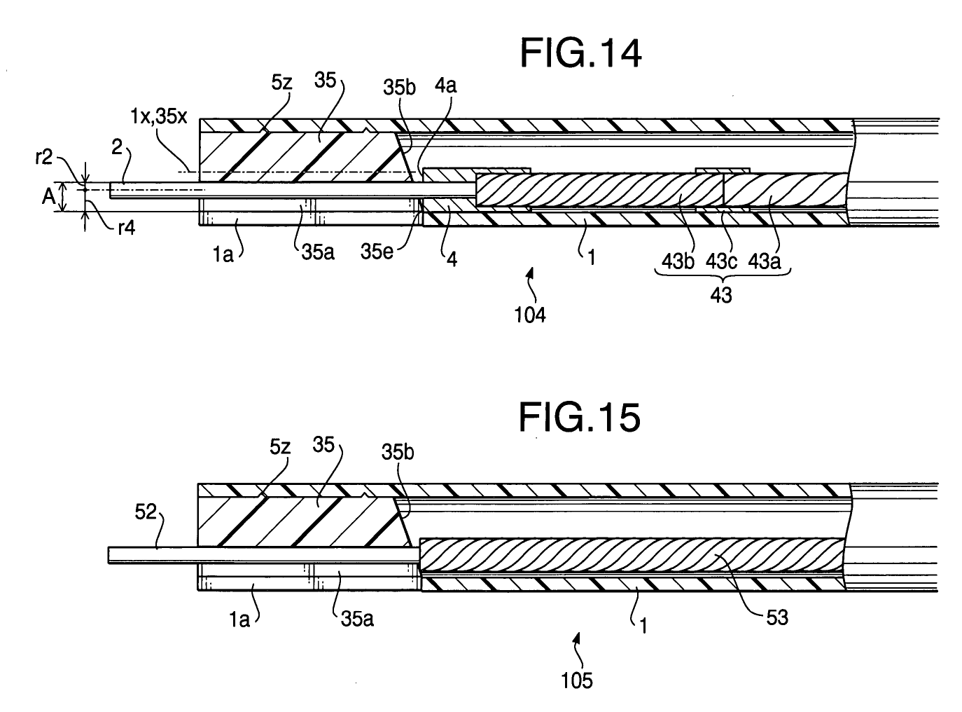

FIG. 14 shows a cross-sectional side view of a distal portion of the treatment tool 104 according to a fourth embodiment of the invention. The operation wire 43 includes a proximal portion 43a, which is formed with a plurality of (for example, seven) thin stainless steel wires twisted to form a single line, and a distal portion 43b, which is formed with a plurality of (for example, thirty-seven or nineteen) thinner stainless steel wires than the wires of the proximal portion 43a twisted to form a single line, and the proximal portion 43a and the distal portion 43b are connected to each other by a joint tube 43c. It should be noted that the distal portion 43b is formed to be more flexible than the proximal portion 43a.

With the above-described operation wire 43, pressing force from the proximal end toward the distal end of the treatment tool 104 is securely conveyed to the distal portion 43b of the operation wire 43, while the distal portion 43b is easily deformed according to an inclination of a rear surface 35b of a stopper member 35 in a lumen 1b of a sheath 1. Thus, an orientation of an electrode 2 can be securely and easily changed.

### Fifth Embodiment

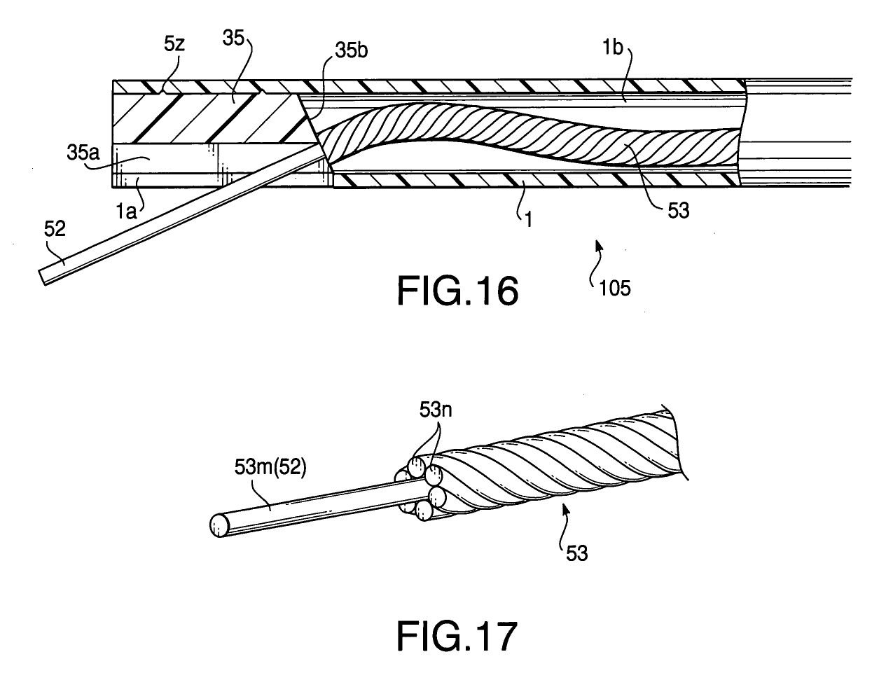

FIG. 15 shows a cross-sectional side view of a distal portion of the treatment tool 105 according to a fifth embodiment of the invention. FIG. 16 shows a cross-sectional side view of the distal portion of the treatment tool 105 with an electrode 52 biased at an angle according to the fifth embodiment of the invention. FIG. 17 shows a perspective view of the electrode 52 and an operation wire 53 of the treatment tool 105 according to the fifth embodiment of the invention.

As shown in FIG. 17, the operation wire 53 is formed with a core wire 53m and a plurality of (for example, six) peripheral wires 53n, which are surroundingly twisted around the core wire 53m. The core wire 53m is elongated independently from the peripheral wires 53n, and the elongated portion is formed to be the electrode 52. The remaining configuration of the treatment tool 105 is similar to the fourth embodiment.

With the above-described configuration, the operation wire 53 is provided with an equivalent resiliency over an entire length thereof. It should be noted that the operation wire 53 does not require a connecting member 4 with rigidity, and can be deformed from a distal portion thereof, which is brought in contact with a rear surface 35a of a stopper member 35 when the operation wire 53 is pressed against the stopper member 35, therefore the orientation of the electrode is effectively changed in relatively small space.

Although an example of carrying out the invention have been described above, the present invention is not limited to the above described embodiments. For example, in the above described embodiment, the rear surfaces 5a, 35a of the stopper members 5, 35 is formed to be inclined, however, the front surface 4a of the connecting member 4 may be formed to be inclined and the rear surfaces 5a, 35a may be formed to be perpendicular to the axis of the sheath 1. Further, the stopper members 5, 35 may be formed integrally with the sheath 1. It should be further noted that the present invention may be applied to a treatment tool that is not supplied with high-frequency electrical current.

The present disclosure relates to the subject matter contained in Japanese Patent Applications No. 2005-032392, filed on Feb. 9, 2005, No. 2005-058475 and No. 2005-058476, filed on Mar. 3, 2005, which are expressly incorporated herein by reference in their entireties.

What is claimed is:

1. A treatment tool for an endoscope, comprising:
a flexible sheath being made of electrically insulating material to be inserted through an instrument channel of the endoscope,
an operation wire, which is adapted to be advanced and retracted in an axial direction of the sheath inside the sheath, and
a treatment instrument, which is arranged at a distal portion of the treatment tool and is adapted to be protruded and retracted from a distal end of the sheath,
wherein the operation wire is connected to the treatment instrument via a connecting member having a distal surface,
wherein the sheath includes a stopper surface at the distal end thereof, the stopper surface being inclined with respect to the distal surface of the connecting member when the connecting member is installed in the sheath,
wherein an orientation of the treatment instrument is changed according to the inclination of the stopper surface by having the distal surface of the connecting member pressed against the stopper surface when the operation wire is pressed from a proximal end of the sheath toward the distal end of the sheath.

2. The treatment tool according to claim 1,
wherein the treatment instrument is formed to be elongated,
wherein the sheath is provided with a stopper member, of which rear surface defines the stopper surface, at the distal end thereof, and
wherein the stopper member is provided with a groove, of which direction corresponds to the orientation of the treatment instrument being changed, so that the treatment instrument is guided in the groove.

3. The treatment tool according to claim 2,wherein a cutout is formed on the distal end of the sheath to be integrated with the groove.

4. The treatment tool according to claim 1, wherein a cross-sectional area of a channel in which the operation wire is arranged is formed to have an elongated shape in parallel with a plane, in which the orientation of the treatment instrument is changed, at least at a distal portion of the sheath so that the operation wire can be deformed therein.

5. A treatment tool for an endoscope, comprising:
a flexible sheath being made of electrically insulating material to be inserted through an instrument channel of the endoscope,
an operation wire, which is adapted to be advanced and retracted in an axial direction of the sheath inside the sheath, and
a treatment instrument, which is arranged at a distal portion of the treatment tool and is adapted to be protruded and retracted from a distal end of the sheath,
wherein the operation wire is connected to the treatment instrument via a connecting member having a distal surface,
wherein the sheath includes a stopper surface at a distal portion thereof, the stopper surface being inclined with respect to the distal surface of the connecting member when the connecting member is installed in the sheath,
wherein the distal portion of the sheath includes a groove, in which the treatment instrument is guided, at a position corresponding to a rear end of the stopper surface with respect to a diameter of the distal portion of the sheath, the rear end of the stopper surface being a closest point to the distal surface of the connecting member,
wherein a depth of the groove is formed to be smaller than a perpendicular length from the rear end of the stopper surface to an axis of the sheath, and
wherein an orientation of the treatment instrument is changed according to the inclination of the stopper surface by having the distal surface of the connecting member pressed against the stopper surface when the operation wire is pressed from a proximal end of the sheath toward the distal end of the sheath.

6. The treatment tool according to claim 5,
wherein the groove is formed to allow the treatment instrument to protrude outwardly from the distal end of the sheath in parallel with the axis of the sheath.

7. A treatment tool for an endoscope, comprising:
a flexible sheath being made of electrically insulating material to be inserted through an instrument channel of the endoscope,
an operation wire, which is adapted to be advanced and retracted in an axial direction of the sheath inside the sheath, and
a treatment instrument, which is arranged at a distal portion of the treatment tool and is adapted to be protruded and retracted from a distal end of the sheath,
wherein the operation wire is connected to the treatment instrument via a connecting member having a distal surface,
wherein the sheath includes a stopper surface at a distal portion thereof, the stopper surface being inclined with respect to the distal surface of the connecting member when the connecting member is installed in the sheath,
wherein an orientation of the treatment instrument is changed according to the inclination of the stopper surface by having the distal surface of the connecting member pressed against the stopper surface when the operation wire is pressed from a proximal end of the sheath toward the distal end of the sheath, and
wherein the operation wire includes a proximal portion and a distal portion that is formed with more flexible wire than a wire forming the proximal portion.

8. A treatment tool for an endoscope, comprising:
a flexible sheath being made of electrically insulating material to be inserted through an instrument channel of the endoscope,
an operation wire, which is formed with a core wire and a plurality of peripheral wires, the plurality of peripheral wires being twisted around the core wire, and is adapted to be advanced and retracted in an axial direction of the sheath inside the sheath, and
a treatment instrument, which is arranged at a distal portion of the treatment tool and is adapted to be protruded and retracted from a distal end of the sheath,
wherein the operation wire includes a distal surface, from which the treatment instrument is extended,
wherein the sheath includes a stopper surface at a distal portion thereof, the stopper surface being inclined with respect to the distal surface of the operation wire when the operation wire is installed in the sheath,
wherein an orientation of the treatment instrument is changed according to the inclination of the stopper surface by having the distal surface of the operation wire pressed against of the stopper surface when the operation wire is pressed from a proximal end of the sheath toward the distal end of the sheath, and
wherein the core wire is elongated at a distal portion thereof to form the treatment instrument.
